(19) United States
(12) Patent Application Publication
(10) Pub. No.: US 2004/0102831 A1
(43) Pub. Date: May 27, 2004

(54) Stent having tapered edges

(76) Inventor: Robert J. Murray III, Santa Rosa, CA (US)

Correspondence Address: MEDTRONIC AVE, INC., 3576 UNOCAL PLACE, SANTA ROSA, CA, 95403, US

(21) Appl. No.: 10/302,503

(22) Filed: Nov. 22, 2002

(51) Int. Cl. A61F 2/06

(52) U.S. Cl. 623/1.11

(57) ABSTRACT

A stent delivery system comprises an inner member and an expandable balloon mounted thereon. The stent has a distal edge and a proximal edge, and at least a portion of the stent has an outer diameter that tapers down towards the distal edge. Each stent segment is comprised of a wire member bent to form a generally cylindrical mesh that includes a plurality of axial bends defining proximal and distal edges of the stent segment. The width and/or outer diameter of the axial bends defining the distal edge and/or the proximal edge may be reduced to provide a low profile.

### TECHNICAL FIELD

This invention relates generally to an implantable stent apparatus, and more particularly, to a low profile, modular stent having tapered or narrowed edges rendering it particularly suitable for the treatment of cardiovascular disease including atherosclerosis.

### BACKGROUND OF THE INVENTION

Cardiovascular disease is a leading cause of death, and as a result, the medical community has devised various methods and devices for the treatment of coronary heart disease including those associated with the complications resulting from atherosclerosis or other forms of coronary arterial closing or narrowing. One such treatment utilized in cases involving atherosclerosis and/or other forms of coronary narrowing is referred to as percutaneous transluminal coronary angioplasty, sometimes simply referred to as angioplasty or PTCA. The objective of this technique is to radially enlarge the lumen of the impacted artery. This is accomplished by positioning an expandable balloon in a target lesion (i.e., the narrowed lumen of the coronary artery). Inflation of the balloon causes (1) soft or fatty plaque deposits to be flattened by the balloon and (2) hardened deposits to crack and split thereby enlarging the lumen. In addition, the artery wall itself is stretched by the inflated balloon.

In a typical percutaneous transluminal coronary angioplasty (PTCA) procedure, a hollow guiding catheter is introduced into the cardiovascular system of a patient via a relatively large vessel such as the femoral artery in the groin area or the brachial artery in the arm. After access to the patient's cardiovascular system has been achieved, a short hollow sheath is inserted to maintain the passageway during the procedure. After the guiding catheter reaches the ostium of the coronary artery to be treated by angioplasty, a flexible guide wire and a dilatation catheter having a balloon on the distal end thereof are introduced into the guide catheter with the guide wire sliding through the dilatation catheter. The guide wire is advanced through a target lesion in the vasculature. A balloon or dilatation catheter (made of, for example, polyethylene, polyethylene terathalate, PEBAX (polyamide block copolymers and polyester block copolymers), polyvinyl chloride, polyolefin, nylon, or other suitable substance) is then advanced over the previously advanced guide wire by sliding it along the guide wire until the dilatation balloon is properly positioned across the target lesion. Radiopaque markers in the balloon portion of the dilatation catheter assist in the positioning of the balloon across the lesion. After proper positioning, the balloon is inflated, generally with a contrast material to permit fluoroscopic viewing during the treatment, so as to enlarge the lumen of the artery. Treatment may require that the balloon be alternately inflated and deflated until satisfactory enlargement has been achieved. The balloon is then deflated to a small profile so that the dilatation catheter may be withdrawn from the patient's vasculature and blood flow resumed through the dilated artery. Unfortunately, after angioplasty procedures of this type, there may occur a restenosis of the artery; i.e. a renarrowing of the treated coronary artery that significantly diminishes any positive results of the angioplasty procedure. In the past, restenosis frequently necessitated repeat PTCA or even more drastic open-heart surgery.

To prevent restenosis and strengthen the target area, various devices have been proposed for mechanically keeping the affected vessel open after completion of the angioplasty procedure. Such mechanical endoprosthetic devices, generally referred to as stents, are typically inserted into the vessel, positioned across the target lesion, and then expanded to keep the lumen clear. A stent is mounted in a compressed state around a deflated balloon, and the balloon/stent assembly maneuvered through a patient's vasculature to the site of a target lesion. The balloon is then inflated causing the stent to be expanded to a larger diameter for placement or implantation in the vasculature. The stent effectively overcomes the natural tendency of the vessel walls of some patients to close back down, thereby permitting an increased flow of blood through the vessel that would not be possible if the stent were not in place.

Many types of stents have been proposed and utilized. One such stent involves a tube of stainless steel wire braid. This tube is positioned on a delivery device, such as a catheter in a compressed state, so as to render the outer diameter of the tube and the delivery device as small as possible. After the stent has been positioned across a target lesion, it is expanded as described previously. Another known stent referred to as a Palmaz stent utilizes a stainless steel cylinder having a number of slits in its circumference resulting in a mesh when expanded. The stainless steel cylinder is delivered to a target lesion by means of a balloon catheter, and once in place, is expanded to an appropriate size by inflating the balloon. A more detailed discussion of the Palmaz stent may be found in U.S. Pat. No. 4,733,665, the teachings of which are hereby incorporated by reference.

Unfortunately, the use of such stents has presented certain difficulties. For example, there is generally a limited amount of securement between the stent and the balloon, and this limited securement is not always adequate to ensure that the stent will remain in its proper position while being advanced through the vasculature to a target lesion. Additionally, the outer surface of the delivery device is uneven because the stent generally extends radially outward beyond the balloon. Thus, the stent may contact a vessel wall and be displaced while the catheter negotiates a narrow vessel. Additionally, if during a coronary intervention, the physician has difficulty crossing a target lesion, it may be necessary to pull the stent delivery system back into the guide catheter. In other cases, the lesion may be heavily calcified requiring a high insertion pressure. Either situation could result in premature displacement of the stent and possible risk to the patient.

It has also been found that as a stent delivery system is advanced through a patient's coronary vasculature, the stent may begin flare and separate from a balloon upon which it was crimped. This is especially true at the proximal and distal ends of the stent. As a result, the stent may extend beyond and protrude from the balloon thus increasing the possibility that the proximal and/or distal edges of the stent will impact a vessel wall or peripheral devices such as the guide catheter.

The use of segmented stents has mitigated some of the problems associated with prior art stents; e.g. inability to conform to vessel shape, lack of sufficient flexibility for advancing through and implantation in vascular anatomy, etc. One such device is shown and described in U.S. Pat. No. 5,817,152 issued Oct. 6, 1998 and entitled “Connected Stent Apparatus”, the teachings of which are hereby incorporated by reference. A single stent is comprised of at least two shorter stent segments which are connected, for example, by welding so as to produce a stent tailored to the length of the stenosis to be treated. Unfortunately, segmented of this type still comprise proximal and distal edges which are susceptible to the above referred flaring problem.

It should therefore be appreciated that it would be desirable to provide a stent that overcomes the drawbacks and limitations of the prior art by tapering the proximal and distal edges of the stent so as to improve its maneuverability through narrow vessels of a patient's vasculature.

### SUMMARY OF THE INVENTION

According to a first aspect of the invention, there is provided a stent delivery system comprising an inner member and an expandable balloon mounted thereon. A stent, having a distal edge and a proximal edge, is mounted around at least a portion of the expandable balloon. At least a portion of the stent has an outer diameter that tapers down towards the distal edge of the stent.

According to a further aspect of the invention, there is provided an endovascular support device comprising at least one wire member bent to form a generally cylindrical mesh. A plurality of axial bends comprises the mesh and defines a proximal edge and a distal edge of the support device. Each axial bend is joined to the adjacent axial bends by a plurality of interconnecting struts. The width of the wire member is reduced in the area of the axial bends that define the distal edge of the support device.

### BRIEF DESCRIPTION OF THE DRAWINGS

The following drawings are illustrative of particular embodiments of the invention and therefore do not limit the scope of the invention, but are presented to assist in providing a proper understanding. The drawings are not to scale (unless so stated) and are intended for use in conjunction with the explanations in the following detailed descriptions. The present invention will hereinafter be described in conjunction with the appended drawings, wherein like reference numerals denote like elements, and;

FIG. 1 is a longitudinal view, partially in cross-section, of a conventional balloon/stent assembly;

FIG. 2 is a longitudinal view illustrating a portion of a four-segment modular stent;

FIG. 3 is a top view illustrating a portion of a stent section comprised of a plurality of axial bends interconnected by a plurality of struts;

FIG. 4 is a top of view of a conventional crown/strut assembly;

FIG. 5 is a side view of the crown/strut assembly shown in FIG. 4;

FIG. 6 is a top view of a crown/strut assembly and in accordance with the teachings of the present invention;

FIG. 7 is a side of the crown/strut assembly shown in FIG. 6; and

FIG. 8 is an isometric view of a single stent segment having a distal edge of a smaller width and outer diameter in accordance with the teachings of the present invention.

### DETAILED DESCRIPTION OF THE EXEMPLARY EMBODIMENT

The following description is exemplary in nature and is not intended to limit the scope, applicability, or configuration of the invention in any way. Rather, the following description provides a convenient illustration for implementing an exemplary embodiment of the invention. Various changes to the described embodiment may be made in the function and arrangement of the elements described herein without departing from the scope of the invention.

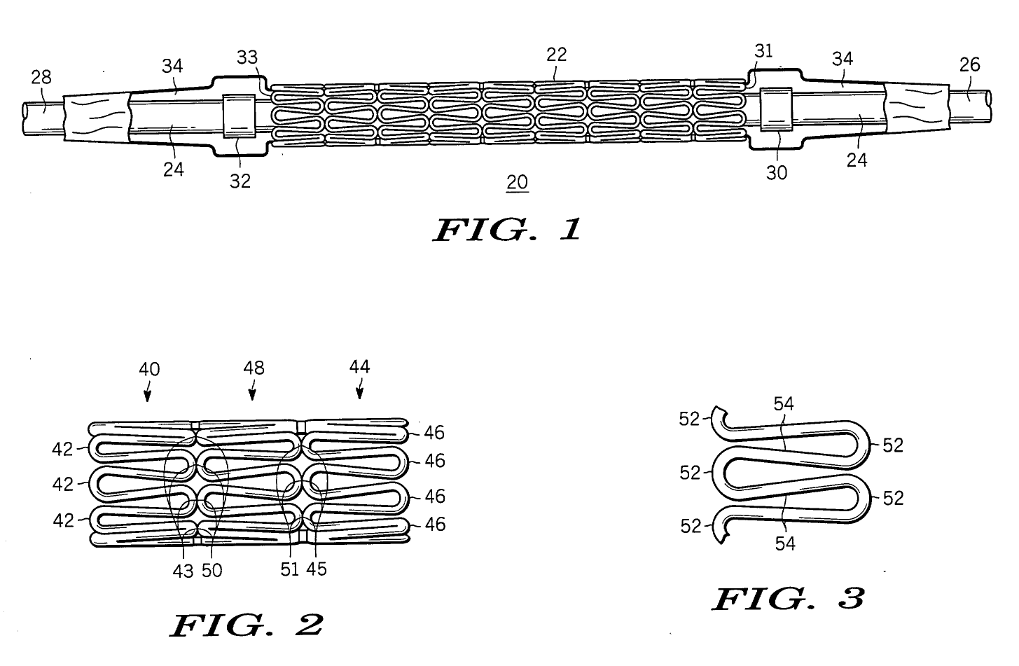

FIG. 1 is a longitudinal, cross-sectional view of a balloon/stent delivery system or assembly comprising an endovascular support device such as a stent 22 having distal and proximal edges 31 and 33 respectively and an opening therethrough, an inner member or wire lumen 24 having a distal end 26 and a proximal end 28, and distal and proximal radiopaque marker bands 30 and 32 respectively which are positioned on inner member or wire lumen 24 near the distal and proximal ends of stent 22. Stent 22 may be of any form or configuration suitable for the intended purpose (e.g. substantially cylindrical), and may comprise one or more stent segments depending on the size and configuration of the vessel to be treated. It will be recognized by those skilled in the art that inner member or guide lumen 24 is configured for the insertion of a conventional guide wire (not shown) which will enable the balloon/stent assembly to be guided to and positioned at a target location in the vessel to be treated.

Any conventional or modified balloon catheter device may be used such as a PTCA balloon catheter. An expandable balloon portion 34 is mounted on inner member 24 in a compressed or collapsed state beneath stent 22 and extends beyond the proximal and distal ends of stent 22. Balloon 34 is generally made of a pliable material such as polyethylene, polyethylene terathalate, PEBAX (polyamide block copolymers and polyester block copolymers), polyvinyl chloride, polyolefin, nylon or the like. The length and the diameter of the balloon may be selected to accommodate the particular configuration of the stent to be deployed. Stent 22 may be constructed of any implantable material having good mechanical strength, such as stainless steel, tantalum, super-elastic nickel-titanium alloys, or high-strength thermoplastic polymers. The outside or wall of the stent may be selectively plated with platinum or other implantable radiopaque substance to provide visibility during fluoroscopy. The cross-sectional shape of the tubular finished stent 22 may be circular, ellipsoidal, rectangular, hexagonal, square, or any other desired shape, although a circular or ellipsoidal cross-section is preferable. The length and width of stent 22 is generally determined to a large degree by the size of the vessel into which the stent will be deployed. Stent 22 must be of sufficient length to extend across a significant portion of the target area while maintaining its axial orientation without shifting under the hydraulics of blood flow, and at the same time not be unnecessarily long so as to result in the introduction of a large amount of material into the vessel.

After stent selection, stent 22 is compressed upon the outside of balloon 34. An inner sheath (not shown) is placed over each end of balloon 34 and an exterior sheath (also not shown), is placed over the ends of the interior sheath so as to cover stent 22 and overlap with the interior sheaths. The assembly is then pressurized by introducing air or an inert gas such as nitrogen through the lumen 24 into the interior of balloon 34 so as to expand the balloon within the sheaths. The assembly is then exposed to an elevated temperature while maintaining pressurization of the balloon. The pressure may be, for example, approximately 70 psi and the temperature approximately 150 degrees Fahrenheit. Following heating, the balloon/stent assembly is allowed to cool within the sheaths, and this cooling sets the shape of balloon 34. The sheaths may then be removed. This process is described in detail in U.S. Pat. No. 5,836,965 entitled “Stent Delivery and Deployment Method” issued Nov. 17, 1998, the teachings of which are hereby incorporated by reference.

Marker bands 30 and 32, which may be viewed through fluoroscopy, assist in positioning the assembly. When the assembly is properly located across a lesion, the balloon may be inflated in a conventional manner. This results in the generally uniform, symmetrical expansion of the stent and balloon. The amount of inflation and thus the amount of expansion of the stent may be varied as dictated by the lesion itself.

As stated previously, it has been found that as a stent delivery system is advanced through a patient's coronary vasculature, the stent may begin flare and separate from a balloon upon which it was crimped. This is especially true at the proximal and distal ends or edges of the stent. As a result, the stent may extend beyond and/or protrude from the balloon thus increasing the possibility that the proximal and/or distal edges will impact the vessel wall or peripheral devices such as the guide catheter.

The potential for flaring at the proximal and distal edges 31 and 33 respectively of stent 22 can be mitigated as described below in conjunction with FIGS. 2-8. FIG. 2 illustrates a modular endovascular support stent 38 comprised of a proximal section 40 having a proximal edge 42 and a distal edge 43, a distal section 44 having a proximal edge 45 and a distal edge 46, and a first intermediate section 48 having a proximal edge 50 and a distal edge 51. It should be appreciated that while a three-segment modular stent has been shown, the number of segments may be chosen to suit a particular purpose or application. Furthermore, the invention is equally applicable to non-modular stents; e.g. laser-cut stents.

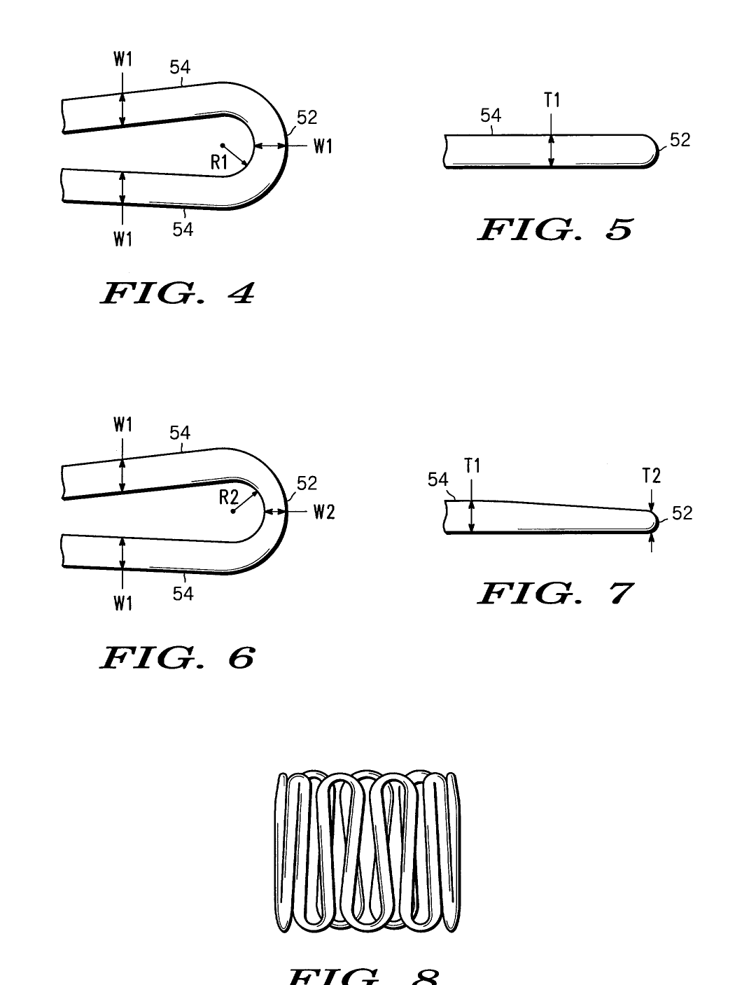

It is generally known that each stent segment may be produced from a machined wire ring or torroid (e.g. machined from stainless steel bar stock) which is then bent or formed into a desired shape (e.g. substantially cylindrical having an opening therethrough), usually through the use of a forming tool. Each stent segment is generally a cylindrical wire mesh tube, and the wire may be formed into a plurality of axial bends or crowns 52 interconnected by a plurality of struts 54 as shown in FIG. 3. Heretofore, the crowns and struts have had a generally circular cross-section of a substantially constant diameter. For example, referring to FIG. 4, which is a top view of a single crown 52 and its adjoining struts 54, it can be seen that the struts and crown have a substantially uniform width W₁. FIG. 5, which is a side view of the crown/strut assembly shown in FIG.4, illustrates that the thickness T₁ of crown 52 and strut 54 is substantially uniform.

FIGS. 6 and 7 are top and side views respectively of a strut and crown assembly in accordance with the teachings of the present invention. As can be seen, the width of struts 54 is caused to taper down and decrease (as, for example, by grinding) as the struts approach crown 52 such that the width of the crown W₂ is significantly less than the strut width W₁ (e.g. W₂ is equal to or less than W₁/2). Furthermore, the radius R2 of crown 52 may be less than radius R1. Similarly, referring to FIG. 7, the thickness of struts 54 begins to taper down or decrease from T₁ at strut 54 to T₂ at the apex of crown 52. Thus, the thickness of crown 52 is substantially less (e.g. fifty percent less) than the thickness of adjoining struts 54. The result is a stent or stent segment having an edge which is narrower and thinner (i.e. has a smaller outer diameter) than the remaining stent or stent segment as is shown in FIG. 8.

Using the above technique, the distal and/or proximal edges of a modular or unitary stent may be tapered in order to create stent edges having a smaller, tighter profile thereby reducing the possibility of flaring. That is, the stent may be crimped on the balloon to a smaller outer diameter at its tapered edge. Tapering the distal edge provides for deeper penetration into smaller openings in the coronary vasculature. Tapering the proximal edge will (1) provide a tighter grip on the balloon and (2) provide a profile that is less likely to catch on obstacles such as calcified lesions, the guide catheter, etc. The resulting structure would comprise a stent having a first outer diameter intermediate its proximal and distal edges and a second smaller outer diameter at its proximal and distal edges.

In the foregoing specification, the invention has been described with reference to a specific embodiment. However, it should be appreciated that various modifications and changes can be made without departing from the scope of the invention as set forth in the appended claims. Accordingly, the specification and figures should be regarded as illustrative rather than restrictive, and all such modifications are intended to be included within the scope of the present invention.

What is claimed is:

1. A stent delivery system, comprising:
an inner member;
an expandable balloon mounted on said inner member; and
a stent mounted around at least a portion of said expandable balloon, said stent having a distal edge and a proximal edge, said stent having a reduced thickness proximate said distal edge.

2. A stent delivery system according to claim 1 wherein said stent has a reduced thickness proximate said proximal edge.

3. A stent delivery system according to claim 2 wherein said stent has a thickness proximate said distal edge and proximate said proximal edge that is less than that intermediate said distal edge and said proximal edge.

4. A stent delivery system according to claim 1 wherein the thickness of said stent proximate said distal edge is reduced by at least fifty percent over the thickness of a remaining portion of said stent.

5. A stent delivery system, comprising:
an inner member;
an expandable balloon mounted on said inner member; and
a stent mounted around at least a portion of said expandable balloon, said stent having a distal edge and a proximal edge, at least a portion of said stent having an outer diameter which tapers down towards said distal edge.

6. A stent delivery system according to claim 5 wherein at least a portion of said stent has an outer diameter which tapers down towards said proximal edge.

7. A stent delivery system according to claim 6 wherein said stent has an outer diameter proximate said distal edge and said proximal edge which is less than that intermediate said distal edge and said proximal edge.

8. A stent delivery system according to claim 7 wherein said outer diameter proximate said distal edge and said proximal edge is at least fifty percent less than the outer diameter of said stent intermediate said distal edge and said proximal edge.

9. An endovascular support device, comprising at least one generally tubular stent section having a wall defining an opening therethrough and having a proximal edge portion, a distal edge portion, and a first section intermediate said proximal edge portion and said distal edge portion, the thickness of said wall in said intermediate section being greater than the thickness of said wall in said distal edge portion.

10. An endovascular support device according to claim 9 wherein the thickness of said wall in said intermediate section is greater than the thickness of said wall in said proximal edge portion.

11. An endovascular support device according to claim 10 wherein said tubular stent section is substantially cylindrical.

12. An endovascular support device according to claim 11 wherein said intermediate section has an outer diameter greater than an outer diameter of said distal edge portion.

13. An endovascular support device according to claim 12 wherein said intermediate section has an outer diameter greater than an outer diameter of said proximal edge portion.

14. An endovascular support device according to claim 13 wherein the outer diameter of said intermediate section tapers down toward said distal edge portion.

15. An endovascular support device according to claim 14 wherein the outer diameter of said distal edge portion tapers down toward a distal edge of said distal edge portion.

16. An endovascular support device according to claim 13 wherein the outer diameter of said intermediate section tapers down towards said proximal edge portion.

17. An endovascular support device according to claim 14 wherein the outer diameter of said proximal edge portion tapers down toward a proximal edge of said proximal edge portion.

18. An endovascular support device, comprising:
a proximal, generally cylindrical stent section having a first proximal edge portion and a first distal edge portion;
a distal, generally cylindrical stent section having a second proximal edge portion and a second distal edge portion; and
at least one, generally cylindrical intermediate stent section coupled between said first distal edge portion and said second proximal portion, said proximal, distal, and intermediate stent sections having a first outer diameter which tapers down to a second outer diameter proximate said second distal edge portion, said second outer diameter being substantially less than said first outer diameter.

19. An endovascular support device according to claim 18 wherein said first outer diameter tapers down to a third outer diameter proximate said first proximal edge portion, said third outer diameter being substantially less than said first outer diameter.

20. An endovascular support device, comprising at least one wire member bent to form a generally cylindrical mesh comprised of a plurality of axial bends that define a proximal edge and a distal edge of said support device, each axial bend joined to adjacent axial bends by a plurality of interconnecting struts, wherein the width of said wire member is reduced in the area of the axial bends defining said distal edge.

21. An endovascular support device according to claim 20 wherein the width of said wire member is reduced in the area of the axial bends defining said proximal edge.

22. An endovascular support device according to claim 20 wherein the width of said wire member is reduced by at least fifty percent.

23. An endovascular support device according to claim 21 wherein an outer diameter of said cylindrical mesh is reduced in the area of the axial bends defining said distal edge.

24. An endovascular support device according to claim 23 wherein an outer diameter of said cylindrical mesh is reduced in the area of the axial bends defining said proximal edge.
